(19) United States
(12) Patent Application Publication
(10) Pub. No.: US 2023/0270117 A1
(43) Pub. Date: Aug. 31, 2023

(54) MICROBIAL COMPOSITION AND METHOD FOR PRODUCING THEREOF FOR USE IN TREATMENT OF CONTAMINATED SOIL, WATER, AND/OR SURFACES

(71) Applicant: Environmental Bioorganic Sciences Corporation, Las Vegas, NV (US)

(72) Inventors: James Rodney Dickerson, Homer, LA (US); Malcolm Burbank, Pullman, WA (US); Valerie Rogers, Pullman, WA (US)

(73) Assignee: Environmental Bioorganic Sciences Corporation, Las Vegas, NV (US)

(21) Appl. No.: 18/015,999

(22) PCT Filed: Jul. 13, 2021
(86) PCT No.: PCT/IB2021/056313

§ 371 Date: Jan. 13, 2023

Related U.S. Application Data
(60) Provisional application No. 63/051,057, filed on Jul. 13, 2020.
(60) Provisional application No. 63/087,799, filed on Oct. 5, 2020.
(60) Provisional application No. 63/104,841, filed on Oct. 23, 2020.
(60) Provisional application No. 63/130,087, filed on Dec. 23, 2020.
(60) Provisional application No. 63/142,804, filed on Jan. 28, 2021.
(60) Provisional application No. 63/142,821, filed on Jan. 28, 2021.

(51) Int. Cl. A01N 63/27; A01N 63/22; A01N 63/20; A01P 1/00; C12N 1/20; C05F 11/08; C02F 3/34; B09C 1/10

(52) U.S. Cl. CPC A01N 63/27; A01N 63/22; A01N 63/20; A01P 1/00; C12N 1/20; C05F 11/08; C02F 3/348; B09C 1/10; C02F 2303/04; C02F 2305/06; C02F 2103/007

(57) ABSTRACT

There is provided microbial compositions and methods for producing thereof and use of compositions thereof in treatment of contaminated soil, water, and/or surfaces. In one aspect, there is provided method for reducing microbial contamination of a microbial contaminated body, the method comprises: inactivating resident vegetative microbiology from an extract obtained from a contaminated of body to inactivate the resident vegetative microbiology in the extract, selecting one or more soil-based microbes suitable for growth in the contaminated body, growing the one or more soil-based microbes with the inactivated extract to allow the one or more soil-based microbes to adapt to the inactivated extract, releasing the one or more soil-based microbes into the contaminated body where the one or more soil-based microbes dominate and reduce microbial contamination of the microbial contaminated body.

### CROSS REFERENCE TO RELATED APPLICATIONS

This application claims the benefit of the priority of U.S. Provisional Patent Application Nos. 63/051,057, filed Jul. 13, 2020; 63/087,799, filed; Oct. 5, 2020; 63/104,841, filed Oct. 23, 2020; 63/130,087, filed Dec. 23, 2020; 63/142,804, filed Jan. 28, 2021; and 63/142,821, filed Jan. 28, 2021, the contents of which are all incorporated herein by reference in their entirety.

### FIELD OF THE INVENTION

The invention relates to microbial compositions and method for producing thereof and use of compositions thereof in treatment of contaminated soil, water, and/or surfaces.

### BACKGROUND OF THE INVENTION

Surface waters microcosms are heathy when there is a balance of the microbiology existing within them consuming organic materials and nutrients without producing toxic metabolites as byproducts, harmful to animals and humans.

Imbalance occurs when the amounts of organic materials and nutrients, together with environmental conditions, favor a disproportionate growth rate of microbiology producing toxins in general, and the species cyanobacteria is the most prevalent.

Cyanobacteria, also known as blue-green algae, are among the oldest microbial life on Earth and are thought to be more than 3,500 million years old. They are thought to be primary source of oxygen in the early atmosphere. A majority of cyanobacteria are aerobic photoautotrophs and require only light, water, CO2, and inorganic compounds. These bacteria are among the very few organisms that can perform oxygenic photosynthesis and respiration in the same compartment. Photosynthesis in cyanobacteria uses the energy of sunlight to split water into oxygen, protons and electrons. While most cyanobacteria use water as an electron donor, some species share with archaea the ability to reduce elemental sulfur, anaerobically, in the dark.

Cyanobacteria are chemically diverse, with the ability to grow over a wide range of conditions and, as such these bacteria are common in soil and water. Some cyanobacteria exist as symbionts of protozoans, diatoms, fungi and plants. Cyanobacteria are often the first microbes to inhabit rocks and soil. Cyanobacteria are named after the bluish pigment, phycocyanin, which is used to capture light for photosynthesis. These bacteria also contain chlorophyll a, the same photosynthetic pigment used by plants. Many species of cyanobacteria can fix elemental nitrogen (N2) under anaerobic conditions, giving them a competitive advantage over may other environmental bacteria in low-nitrogen environments. They are among just a few organisms that can oxidize N2 to nitrite or nitrate and reduce N2 to ammonium.

Cyanobacteria are necessary in a healthy surface water environment; however, chemical fertilizers used in modern agriculture have managed to push the conditions in water bodies that favors these bacteria, resulting in large toxic “blooms” where toxins are released into the water and air that kill higher life forms, including fish, animals, and humans. Nitrogen and phosphorus from over fertilization is the main factor that leads to cyanobacteria blooms in bodies of water so controlling the flow of nutrients into the body of water is a critical step to controlling the cyanobacterial blooms. This process tackles the problem identified by Smith by treating impacted water, including, where possible, close to the nutrient point source, using active microbiology to remove or reduce the nitrogen and phosphorus so that cyanobacteria downstream are deprived of the high concentrations of phosphorus and nitrogen that they need to dominate.

What is needed is a means to restore equilibrium or balance to these water bodies using the process of competitive exclusion. Dickerson taught in U.S. Pat. Nos. 5,578,211 and 5,578,841 that soil microbiology, comprised of ubiquitous soil bacteria could be used to both modify and sustain modification of the sewer and wastewater treatment plant microbiome using the principle of competitive exclusion. In '211 and '84, the process deals with the levels of organics and nutrients within the entire wastewater system. The addition of soil microbiology as practiced in '211 and '841 concerned changing and sustaining the change in the microcosm by the continual addition of a specific formulation of select microbiology in strategic locations in the outer reaches of the sewer system.

The microcosm modification in the outer reaches was shown to grow downstream and, over time, change the entire microbiome within the wastewater treatment facility. It was demonstrated that when a correct number of microbes in an inert spore state were added, the balance could be shifted and sustained. In order to make the process commercial, the microbiology had to be concentrated to extremely high levels so it could be delivered in many locations in an economical fashion on a continual basis.

The primary competitors of cyanobacteria are soil bacteria. When the soil bacteria are not present in populations sufficient to prevent the phosphorous and nitrogen from reaching high levels, cyanobacteria prevail.

What is needed is a means of generating large populations of soil microbiology and continuously adding them to the surface waters in an active state to out-compete the cyanobacteria.

Food producing animals (e.g. cattle, chickens, pigs and turkeys) are the primary source of foodborne pathogens. Pathogens such as Campylobacter species, non-Typhi serotypes of Salmonella enterica, Shiga toxin-producing strains of Escherichia coli, and Listeria monocytogenes are present in irrigation water from various sources of contamination such as animal manure. Applying pathogen contaminated irrigation water contaminates both plant surfaces and soil.

Growing food plants in contaminated soils and applying contaminated irrigation water presents a clear and present danger to human health when these plants are eaten in a raw or undercooked.

Regulatory agencies have processes and procedures in place to minimize the risk of contamination from farm workers and those who handle, package and transport the produce. Nevertheless, it has become more common for outbreaks of pathogens and contaminated produce production with rising frequency.

The consequences of such outbreaks impact on human and animal health sometimes results in death, and the costs of recalls and destruction of produce, and the legal responsibility which may involve both civil damages and, potentially, criminal charges.

While there are many methods for killing and/or removing pathogens from irrigation water, simply removing pathogens from irrigation water does not solve the problem with soils that have been contaminated after years of application of contaminated water.

It is well established that applying all techniques for disinfecting water using anything other than extreme heat or irradiation carries the potential for some bacteria to survive. Even ultrafiltration may have a small amount of barrier imperfections where a few microbes may get past the barrier.

Irradiation is expensive and complex. Application of extreme heat is well known to kill pathogens and is the obvious choice. When followed by ultrafiltration the complete removal of all microbial pathogens can be achieved.

The use of chemical fertilizers is well established to cause harm to the soil microbiome and the use of chemical disinfectants is comparable to chemical warfare in that it is indiscriminate in killing.

What is needed is a method to both remove pathogens from irrigation water and, simultaneously, restore the soil by killing the pathogens existing in contaminated soils while building a natural resistance to future contamination.

Sustainability in agriculture has become the primary focus of new agricultural technologies for more than a decade. It is now widely accepted the soil microbiome content has been damaged by chemical fertilizers, insecticides, and fungicides. Fertilizers and other chemicals damage the soil microbiome by reducing the microbial populations, which in turn increases the demand for more chemicals. Thus, an unintended consequence of applying fertilizer is that it kills microbes in the immediate soil where applied and results in a cycle of excess nutrients (e.g. excess phosphate that is not bioavailable to plants) that, in turn, enter the runoff of surface waters. In particular, phosphorous has become a problem in runoff as well as accumulated in soil, impacting surface waters causing harmful cyanobacteria outbreaks including damage to final waters like the Gulf of Mexico.

It is known to modify and sustain the modification of the sewer biofilm using the process of competitive exclusion. The continual addition of appropriate amounts of microbiology in appropriate locations proved the efficacy of the process and the numerous benefits obtained from the successful modification.

The soil microbiome is not unlike the biofilm of the sewer system with respect to the wide variety of microbial species competing for food and resources. It has been shown that frequent application of chemicals negatively impacts this microbiome. Application of various, specific, soil microbial strains has shown positive impacts upon plant growth and health; however, farmers using these inputs incur high application costs which restricts both the amount and frequency of applications. Such restrictions obviously impact the results reducing the effectiveness including crop protection and increasing crop yields.

What is needed is a way to restore the agricultural soil microbiome that is sustainable because current practices involve producing specific strains of microbes in a commercial production facility, packaging, storing inventory and transporting to farms for application at very high costs to farmers. The results from such methodology are seldom cost justified to the farmers. Additionally, the current methodologies usually require significant capital cost investment on the part of the farmers.

Foot rot in cattle is a sub-acute or acute, highly infectious disease of the hoof of dairy cows, beef cattle, sheep and goats. The disease often reduces the average weight gain of infected cattle from 2.76 pounds per day to 2.3 pounds per day. Approximately 20% of lameness in cows and cattle is caused by foot rot 1. The most frequent mode of transmission for the disease is when the bacteria from infected livestock come into contact with the hoofs of uninfected livestock.

The disease is most often associated with Fusobacterium necrophorum with secondary infections by Porphyromonas, levii, Staphylococcus aureus, Escherichia coli and Truperella pyogenes. The co-infections are believed to reduce the dose of F. necrophorum required for infection. All of the bacteria commonly associated with foot rot are susceptible tetracycline and streptomycin antibiotics. Tetracyclines and streptomycin are bacteriostatic antibiotics that inhibits protein synthesis and stops bacteria from dividing.

Many dairy operations use foot baths containing copper sulfate to reduce the transmission of the bacteria by removing manure from the cattle's hoof and to kill any bacteria on the exposed surface of the hoof. Copper sulfate inevitably gets combined with the feces of the cattle and affects the downstream processes that deal with the cow manure. Copper sulfate is highly toxic, even in low concentrations to a wide range of bacteria. Unfortunately, copper sulfate is also toxic to environmental bacteria and can accumulate in the lagoons, digesters and on soil if the copper contaminated manure is land applied. Copper contamination of digesters that are used to degrade the cattle manure is toxic to the gut microbe of the cow that are responsible for the anaerobic degradation of the cattle waste and effectively poisons the anaerobic digester, slowing or stopping the degradation of manure.

Members of the genus Bacillus are widely distributed in soil and water and play an important role in recycling/degrading organics, however organics that are contaminated with copper can kill or force into sporulation members of Bacillus and similar genera.

One example is the digestion of copper contaminated dairy waste. One method to degrade dairy waste (manure) is to digest the waste in anaerobic digester utilizing the native gut microbes to degrade the waste. However, many dairy operations utilize copper sulfate foot baths that cattle are walked through to reduce the incident of foot (hoof) rot. The copper gets combined with manure when the manure is scrapped from the dairy floor and the copper contaminated dairy waste is put into digester. Frequently, the copper concentrations are high enough to effectively stop the digestion and the waste backs up.

Accordingly, there is needed a method that increases copper resistance in Bacillus and similar genera and restores their ability to digest contaminated waste.

Toxic metals and organics are present in water that has been impacted by coal fly and bottom ash, water impacted by mining, landfill leachates and other waters.

Metals can be removed by a variety of methods, but the use of passive microbial based removal are often the most accepted due to their low energy costs. Some examples of biologically mediated methods to remediate contaminating metals include 1) biochemical reactor (BCR) where metals are precipitated by sulfate reducing bacteria (SRB) as metal-sulfides, 2) anaerobic reduction (REDOX reaction to change the oxidation state) of metals by anaerobic or facultative bacteria and, 3) a relatively new approach, microbial induced calcite precipitation (MICP), where indigenous or exogenous ureolytic bacteria precipitate the mineral calcite and co-precipitate or sorb metals, reducing their solubility in soils and creating a solid mass.

Accordingly, there is a need for a method that is efficient at decontaminating all metals and/or organic contaminants.

### SUMMARY OF THE INVENTION

In one embodiment, the present disclosure relates to microbial compositions and method for producing thereof for use in treatment of contaminated soil, water, and/or surfaces

In one embodiment, there is provided microbial compositions and methods for producing thereof and use of compositions thereof in treatment of contaminated soil, water, and/or surfaces. In one aspect, there is provided method for reducing microbial contamination of a microbial contaminated body, the method comprises: inactivating resident vegetative microbiology from an extract obtained from a contaminated of body to inactivate the resident vegetative microbiology in the extract, selecting one or more soil-based microbes suitable for growth in the contaminated body, growing the one or more soil-based microbes with the inactivated extract to allow the one or more soil-based microbes to adapt to the inactivated extract, releasing the one or more soil-based microbes into the contaminated body where the one or more soil-based microbes dominate and reduce microbial contamination of the microbial contaminated body.

In one embodiment, there is provided a method for reducing cyanobacteria in a cyanobacteria-contaminated of a body water, the method comprises:
inactivating resident vegetative microbiology from an extract obtained from a cyanobacteria-contaminated of body of water to inactivate the resident vegetative microbiology in the extract,
selecting one or more soil-based microbes suitable for growth in the cyanobacteria-contaminated body of water,
growing the one or more soil-based microbes with the inactivated extract to allow the one or more soil-based microbes to adapt to the inactivated extract,
releasing the one or more soil-based microbes into the cyanobacteria-contaminated body of water where the one or more soil-based microbes dominate and reduce cyanobacteria of the cyanobacteria-contaminated body of water.

In one aspect, the inactivating is by pasteurizing, irradiating, chemically treating and/or mechanically treating the extract.

In one aspect, the soil-based microbes are capable of degrading toxins from the cyanobacteria and/or are facultative spore-formers with rapid growth rates.

In one aspect, the soil-based microbes are from Micrococcaceae, Bacillaceae, Pseudomonadaceae, Planococcaceae, or Cellulomonadaceae.

In one aspect, the releasing is when the one or more soil-based microbes are in a vegetative or active form and capable of assimilating nutrients or chemicals at the cyanobacteria-contaminated body of water.

In one aspect, the releasing is when the metabolism of the one or more soil-based microbes are most active and in the highest density such that when released into the cyanobacteria-contaminated body of water, the one or more soil-based microbes will require and will consume any nutrients in the cyanobacteria-contaminated of body of water.

In one embodiment, there is provided a method for decontaminating irrigation water and restoring soil contaminated with contaminated irrigation water, the method comprises:
inactivating contaminated irrigation water to inactivate vegetative microbiology to produce inactivated irrigation water,
selecting one or more microbes suitable for outcompeting microbiology in the soil contaminated with the inactivated irrigation water,
growing, under aerobic conditions, the one or more microbes with the inactivated irrigation water to allow the one or more microbes to adapt to the inactivated irrigation water and to inactivate any obligate anaerobic bacteria endospores that may have survived inactivation,
releasing the one or more microbes and the inactivated irrigation water into the soil contaminated with contaminated irrigation water where the one or more soil-based microbes dominate and reduce contamination and restore the soil.

In one aspect, the inactivating is by pasteurizing, irradiating, chemically treating and/or mechanically treating the extract.

In one aspect, the method further comprises filtering and/or centrifugation before the growing.

In one aspect, the one or more microbes are soil-based microbes.

In one aspect, the soil-based microbes will promote composting of cellulosic materials in the soil and/or accelerate the breakdown to make additional carbon and nutrients available to the plants.

In one aspect, the one or more microbes are from Micrococcaceae, Bacillaceae, Pseudomonadaceae, Planococcaceae, or Cellulomonadaceae.

In one aspect, the one or more microbes are one or more of nitrogen-fixing microbes, endophytic microbes, and have the ability to make excess phosphate bioavailable to plants.

In one embodiment, there is provided a biofertilizer composition for soil inoculation and/or foliar application, the composition produced according to a method comprising:
macerating an extract of agricultural by-waste,
inactivating the extract to inactivate resident vegetative microbiology in the extract,
flowing inactivated extract into a holding reservoir and a portion of the inactivated extract into a culture reservoir,
growing in the culture reservoir one or more soil-based microbes with the portion of the inactivated extract to allow the one or more soil-based microbes to adapt to the inactivated extract,
flowing a portion of the one or more adapted soil-based microbes when the one or more adapted microbes are in a vegetative or active form and capable of assimilating nutrients or chemicals into the holding reservoir until the concentration of the one or more adapted soil-based microbes in the holding reservoir is from about 10e6 and 10e9 colony forming units (cfu)/ml.

In one aspect, the method further comprises:
flowing out of the holding reservoir the one or more adapted soil-based microbes when the concentration of the one or more adapted soil-based microbes in the holding reservoir is from about 10e6 and 10e9 cfu/ml,
seeding an amount of the one or more soil-based microbes into the culture reservoir so as to allow the seeded amount of the one or more soil-based microbes to adapt to conditions in the culture reservoir, and
flowing an additional portion of the pasteurized extract into the culture reservoir.

In one aspect, the inactivating is pasteurizing, irradiating, chemically treating and/or mechanically treating the extract.

In one aspect, the inactivating is by pasteurizing by elevating the temperature of the extract to not less than about 165 F and not higher than about 212 F.

In one aspect, once the temperature of the extract not less than about 165 F and not higher than about 212 F, maintaining the temperature of the extract for up to about 60 seconds.

In one aspect, once the temperature of the extract not less than about 165 F and not higher than about 185 F, maintaining the temperature of the extract for up to about 60 seconds.

In one aspect, after the pasteurizing and before the flowing into the culture reservoir, reducing the temperature of the extract from to about 105 F or to about 100 F.

In one aspect, the one or more soil-based microbes is one or more of Pseudomonas fluorenscens, B. subtilis, and B. megaterium.

In one aspect, the one or more soil-based microbes is from Micrococcaceae, Bacillaceae, Pseudomonadaceae, Planococcaceae, or Cellulomonadaceae.

In one aspect, the one or more soil-based microbes is copper adapted.

In one embodiment, there is provided a use of the composition to inoculate soil and/or apply to foliage to restore an agricultural microbiome.

In one aspect, the use restores the agricultural microbiome of an infected banana or other food crops.

In one aspect, the infected banana or other food crops is infected with F. oxysporum f. sp. cubense or other plant pathogen or parasitic roundworm.

In one aspect, the parasitic roundworm is nematode.

In one embodiment, there is provided a method for treating foot rot afflicted livestock, the method comprising:
contacting a lower extremity of foot rot afflicted livestock with a microbial composition, the composition comprises at least one microbe adapted to degrade livestock manure and/or produce a bacteriostatic antibiotic.

In one aspect, the at least one microbe is a facultative anaerobic microbe.

In one aspect, the at least one microbe is a soil-based microbe.

In one aspect, the at least one microbe is a plurality of microbes comprising a first microbe adapted to degrade the livestock manure and a second microbe adapted to produce the bacteriostatic antibiotic.

In one aspect, the at least one microbe is from the genus Bacillus.

In one aspect, the at least one microbe is a copper adapted microbe.

In one aspect, the copper adapted microbe is a copper adapted Bacillus.

In one embodiment, there is provided a microbial composition for treating foot rot afflicted livestock, the composition comprises:
at least one microbe adapted to degrade livestock manure and/or produce a bacteriostatic antibiotic.

In one aspect, the at least one microbe is a facultative anaerobic microbe.

In one aspect, the at least one microbe is a soil-based microbe.

In one aspect, the at least one microbe comprises a first microbe adapted to degrade the livestock manure and a second microbe adapted to produce the bacteriostatic antibiotic.

In one aspect, the at least one microbe is a copper tolerant microbe.

In one aspect, the copper adapted microbe is a copper adapted Bacillus.

In one embodiment, there is provided a method of producing copper tolerant microbes adapted to degrade copper contaminated organic waste, the method comprises:
culturing copper intolerant microbes in a solid growth medium containing a base copper level at about 35 degrees for an incubation time of about 6 to about 24 hours,
selecting the base copper level tolerant microbes and growing the base copper level tolerant microbes in a liquid nutritional medium,
harvesting at least a portion of the base copper level tolerant microbes and growing the at least a portion of the base copper level tolerant microbes in a solid growth medium containing an elevated copper level at about 35 degrees for an incubation time of about 6 to about 24 hours,
obtaining copper tolerant microbes by selecting the elevated copper tolerant microbes.

In one aspect, the method further comprises, prior to the culturing of the copper intolerant microbes in the solid growth medium containing the base copper level: reconstituting freeze-dried copper intolerant microbes, and inoculating liquid nutritional medium with the copper intolerant microbes.

In one aspect, the method further comprises, after the selecting the elevated copper tolerant microbes: growing the elevated copper tolerant microbes in a MnCl₂-supplemented nutritional medium to sporulation.

In one aspect, the base copper level is from about 12 ppm to less than about 30 ppm copper and the elevated copper level is from at least 30 ppm.

In one aspect, the liquid nutritional medium comprises Difco Nutrient Broth, the solid growth medium comprises agar, and MnCl₂-supplemented nutritional medium comprises 0.1 M MnCl₂.

In one aspect, the copper intolerant microbes are from Micrococcaceae, Bacillaceae, Pseudomonadaceae, Planococcaceae, or Cellulomonadaceae.

In one aspect, the copper intolerant microbes are B. subtilis subsp. Inaquosorum, B. subtilis 6051a, B. licheniformis, B. mojavensis, or B. megaterium.

In one embodiment, there is provided copper tolerant microbes adapted to degrade copper contaminated organic waste produced according to a method that comprises:
culturing copper intolerant microbes in a growth medium containing a base copper level at about 35 degrees for an incubation time of about 6 to about 12 hours,
selecting the base copper level tolerant microbes and growing the base copper level tolerant microbes in a liquid nutritional medium,
obtaining at least a portion of the base copper level tolerant microbes and growing then at least a portion of the base copper level tolerant microbes in a growth medium containing an elevated copper level at about 35 degrees for an incubation time of about 6 to about 12 hours,
obtaining copper tolerant microbes by selecting the elevated copper level tolerant microbes.

In one aspect, the method further comprises, prior to the culturing of the copper intolerant microbes in the solid growth medium containing the base copper level: reconstituting freeze-dried copper intolerant microbes, and
inoculating liquid nutritional medium with the copper intolerant microbes.

In one aspect, the method further comprises, after the selecting the elevated copper tolerant microbes: growing the elevated copper tolerant microbes in a MnCl₂-supplemented nutritional medium to sporulation.

In one aspect, the base copper level is from about 12 ppm to less than about 30 ppm copper and the elevated copper level is from at least 30 ppm.

In one embodiment, there is provided a use of the copper tolerant microbes for degrading copper contaminated organic waste.

In one embodiment, there is provided a method for remediating metal-impacted water and/or remediating organic contaminated water, the method comprises:
flowing metal-impacted water and/or organic contaminated water to an aerated fluidized bed reactor comprising sand and soil-based bacteria to reduce susceptible metals in the water and/or hydrocarbon degrading bacteria to reduce organic contaminants in the water,
retaining for a first period sufficient to allow the soil-based bacteria to reduce susceptible metals in the water and/or the hydrocarbon degrading bacteria to remove organic contaminants in the water, and form treated water.
flowing clean water, when the treatment time has lapsed, to the first fluidized bed to displace the treated water,
flowing the displaced treated water containing any susceptible metals not reduced in the aerated fluidized bed reactor and/or organic contaminants not removed in the aerated fluidized bed reactor to an anaerobic fluidized bed reactor comprising sand and anaerobic or facultative bacteria,
retaining for a second period sufficient to allow the anaerobic or facultative bacteria to reduce any susceptible metals not reduced in the aerated fluidized bed reactor and/or remove organic contaminants not removed in the aerated fluidized bed reactor, and form remediated metal-impacted water and/or remediated organic contaminated water.

In one aspect, the method further comprises:
removing the sand from the aerated fluidized bed reactor and/or anaerobic fluidized bed reactor before the sand agglomerates and/or forms a solid mass.

In one aspect, the method further comprises:
solidifying the removed sand.

In one aspect, the method further comprises:
providing at least one additional fluidized bed reactor.

In one aspect, the aerated fluidized bed reactor comprises microbial induced calcite precipitation (MICP).

In one aspect, the soil-based bacteria is ureolytic bacteria to precipitate the mineral calcite and/or co-precipitate or sorb the susceptible metals.

In one aspect, the MICP co-precipitates Ca2+, Cu2+, Zn2+, Mg2+Mn2+Cd2+, Co2+, Ni2+, Zn2+, Pb2+, Fe2+, As, Cr, other cations, or radionuclides.

In one aspect, the soil-based bacteria is sulfate reducing bacteria (SRB).

In one aspect, the SRB precipitates Cu2+, Fe2+, Zn2+, Ni2+, Cd2+.

In one aspect, the SRB precipitate the susceptible metals as metal sulfides.

In one aspect, the aerated fluidized bed reactor reduces Hg, Se, As, and/or removes poly- and per-fluorinated (PFAS) compounds.

In one aspect, the method reduces nitrates, sulfates, and susceptible metals.

In one aspect, the method reduces nitrogen, sulfur, and phosphorous.

In one aspect, the soil-based bacteria, hydrocarbon degrading bacteria, or the anaerobic or facultative bacteria are from either indigenous or exogenous microbial sources.

In one aspect, the metal-impacted water and/or the organic contaminated water are as a result of water impacted by coal fly and bottom ash, mining, or landfill leachates.

### BRIEF DESCRIPTION OF THE DRAWINGS

FIG. 1 is a flow diagram of the process for reducing cyanobacteria in a cyanobacteria-contaminated of a body water according to an embodiment of the present invention;

FIG. 2 is a flow diagram of the process for decontaminating irrigation water and restoring soil contaminated with contaminated irrigation water according to an embodiment of the present invention;

FIG. 3 is a flow diagram of the process for producing a biofertilizer composition for soil inoculation and/or foliar application according to an embodiment of the present invention;

FIG. 4 is a flow diagram of the process for producing copper tolerant microbes adapted to degrade copper contaminated organic waste according to an embodiment of the present invention;

FIG. 5 is a graph showing the results of a Total Solids digestion over time study at 5:1 of copper tolerant microbes adapted to degrade copper contaminated organic waste according to an embodiment of the present invention;

FIG. 6 is a graph showing the results of a Total Solids digestion over time study at 10:1 of copper tolerant microbes adapted to degrade copper contaminated organic waste according to an embodiment of the present invention;

FIG. 7 is a photo showing 6 dried foils from the results of study at 10:1 at 0, 2, 8, 24, 48, and 72 hours; and

FIG. 8 is a flow diagram of a process for remediating metal-impacted water and/or remediating organic contaminated water according to an embodiment of the present invention.

### DETAILED DESCRIPTION OF THE INVENTION

### Definitions

The terminology used herein is for the purpose of describing embodiments only and is not intended to be limiting of the invention.

As used herein, the singular forms “a”, “an” and “the” are intended to include the plural forms as well, unless the context clearly indicates otherwise.

Furthermore, to the extent that the terms “including”, “includes”, “having”, “has”, “with”, or variants thereof are used in either the detailed description and/or the claims, such terms are intended to be inclusive in a manner like the term “comprising.”

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

The term “about” or “approximately” means within an acceptable error range for the value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where values are described in the application and claims, unless otherwise stated the term “about” meaning within an acceptable error range for the value should be assumed.

“Optional” or “optionally” means that the subsequently described event or circumstance can or cannot occur, and that the description includes instances where the event or circumstance occurs and instances where it does not.

Detailed embodiments of the instant invention are disclosed herein, however, it is to be understood that the disclosed embodiment is merely exemplary of the invention, which may be embodied in various forms and is in no way intended to limit the invention, its application or uses. Therefore, specific composition ranges disclosed herein are not to be interpreted as limiting, but merely as a basis for the claims and as a representation basis for teaching one skilled in the art to variously employ the present invention in virtually any appropriately detailed composition. The embodiments of the invention may be practiced without the theoretical aspects presented. Moreover, the theoretical aspects are presented with the understanding that the Applicant does not seek to be bound by the theory presented. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

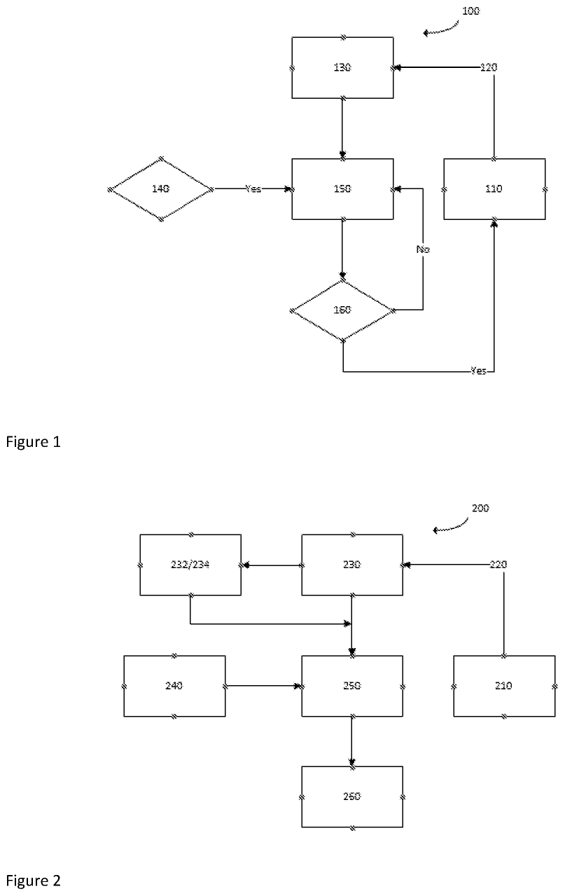

With reference to FIG. 1, according to an embodiment, there is provided a method 100 for reducing cyanobacteria in a cyanobacteria-contaminated of a body water 110.

The system uses target water 120 for rapid growth of the complimentary microbiology that first pasteurizes 130 the water to remove competing microbiology. In one aspect, samples of target water 120 are obtained and examined for microbiology content and ability to grow complimentary because competitive bacteria are already present.

In one aspect, there is provided a supplement to the indigenous population with complimentary bacteria 140 prevent cyanobacteria from dominating.

In one aspect, the bacteria 140 is selected with specific characteristics for competition and/or be adapted to thrive in the target surface waters. The selected bacteria 140, would in aspects, be facultative spore-formers with rapid growth rates such as the naturally occurring, ubiquitous strains from the genus Bacillus.

Additionally, the selected bacteria 140 can be capable of degrading toxins from the cyanobacteria that may be released in the course of the process.

At the growth step 150, the microbes 140 are grown in the presence of the pasteurized extract of target water 120. At step 160, an assessment of the growth of the microbes 140 is done to determine if the microbes 140 are into a vegetative (active) form, capable of assimilating the target nutrients or chemicals, and that they are being delivered into the environment when their metabolism is most active as they start to transition from exponential growth to starvation.

In one aspect, “active microbiology” in the present means that In one aspect, the microbes 140 have eaten all of the food available in our growth system and face starvation. In that state, there is a maximum cell density that desperately need food and will rapidly consume any nutrients in the target water/soil/system.

Thus, in one embodiment, the process 100 provides supplemental bacteria to be grown onsite in large populations for continual addition.

In a further aspect, any additional nutrient materials that may be required to facilitate rapid growth after pasteurization can be identified in the testing laboratory. In particular, some laboratory testing could be done to determine the formulations and amounts that can be added to achieve sustainable domination and suppress the cyanobacteria, preventing the blooms.

In some aspects, these generator systems will be lower energy input to make solar power possible because they should be in the upper reaches of the tributaries and, like in sewer systems, will grow and dominate the downstream receiving bodies.

With reference to FIG. 2, according to an embodiment, there is provided a method 200 for decontaminating irrigation water and restoring soil contaminated with contaminated irrigation water. The methodology described herein accomplishes both removal of contaminants from irrigation water 210 for use in restoration of the soil, together will the added benefit of promoting plant growth and health, without removing significant amounts nitrogen nutrients that may be contained in the water and replacing with nitrogen-fixing bacteria.

While predatory microbiology (e.g. bacteria and viruses) can specifically target pathogens; however, no one before now has devised a methodology that will produce sufficient populations of predatory microbes in situ at a sustainable cost for application to large areas of farmland.

The process 200 described herein not only accomplishes the sustainability requirements, but also adds the additional benefit of supplying plant growth and health promoting bacteria to the disinfected irrigation water together with the pathogen predators.

The combined beneficial impact of applying the treated irrigation water is to remediate the soil microbiome by removing pathogens and providing microbiology that promotes plant growth and health, as well as reducing the chemical fertilizer inputs. The combined benefits offset the cost of the novel methodology for removing pathogens from irrigation water and soils.

In one embodiment, the process 200, in general terms, is a source 210 of contaminated irrigation water 220 and at step 230 the pathogen contaminated irrigation water 220 is pasteurized. Optionally, after the pasteurization step 230 are steps of particulate removal 232, then ultrafiltration 234 to remove any remaining bacteria and pathogens. This will result in a nutrient rich water that can then be used to grow predatory bacteria 240 specific to pathogens and/or bacteria that can stimulate plant growth and reduce plant predation by plant pathogens/pests.

In one aspect, the process uses of aeration in the growth of the pasteurized, separated and lysed bacteria stream to prevent the growth of pathogenic strict anaerobic bacteria endospores that survive the pasteurization process. The endospores of obligate anaerobic soil pathogens are activated by the pasteurization process and will become vegetative. Aeration of the pasteurized media provides oxygen which is fatal to the obligate anaerobic vegetative cells that germinate during the pasteurization process.

Additionally, the aerobic state in the growth process supports a faster growth rate of the facultative anaerobic and supports obligate aerobic soil bacteria that are cultured to inoculate the soil as predatory against pathogens and that provide protection/promote growth for crops.

Keeping energy input to a minimum is of importance to the overall sustainable economy of the process, therefore heat recovery can be used as a mean to pre-heat the flow with additional energy input supplied upstream to achieve target pasteurization temperature. Thus in one aspect, the tank is operated such that any anaerobic pathogenic spores that are activated are killed by aeration. The pasteurized flow leaving the heating process can be cooled with the pre-pasteurization flow being regulated to achieve a target temperature of less than 104 F.

At a growth step 250, the high populations of predatory and plant growth and health promoting bacteria 240 are grown in batches mixed with the pasteurized water before it is sent to irrigation equipment 260.

In one aspect, the ideal growth media for the predatory and plant growth and health promoting bacteria is the nutrients released from bacteria lysed in the pasteurization process, and the application of aeration (oxygen) to kill anaerobic pathogenic bacteria grown of spores that survive and are activated by the heat in the pasteurization process.

The specialized predatory and plant growth and health promoting bacteria are grown to high populations in the least expensive manner in situ with minimal energy input which means a liberal application rate on a regular basis to soil can be made inexpensively, thereby achieving a state of competitive exclusion in the soil. Such domination in the soil is necessary to prevent undesired bacteria from returning as it is well known the soil microbiome is a battleground with competing microbiological entities.

The formulation of plant growth and health promoting bacteria can be comprised of any number of non-pathogenic soil microbiology, selected for the specific application and crop. And important aspects of this microbiology are the ability to fix nitrogen as well as endophytic attributes and the ability to discourage harmful nematodes from inhabiting the treated soil, and the ability to make the excess phosphate—caused by years of chemical fertilizer application—bioavailable to the plants.

In one aspect, the composition destined for use for irrigation of the contaminated soil also contains soil bacteria that promote composting of cellulosic materials in the soil, accelerating the breakdown to make additional carbon and nutrients available to the plants.

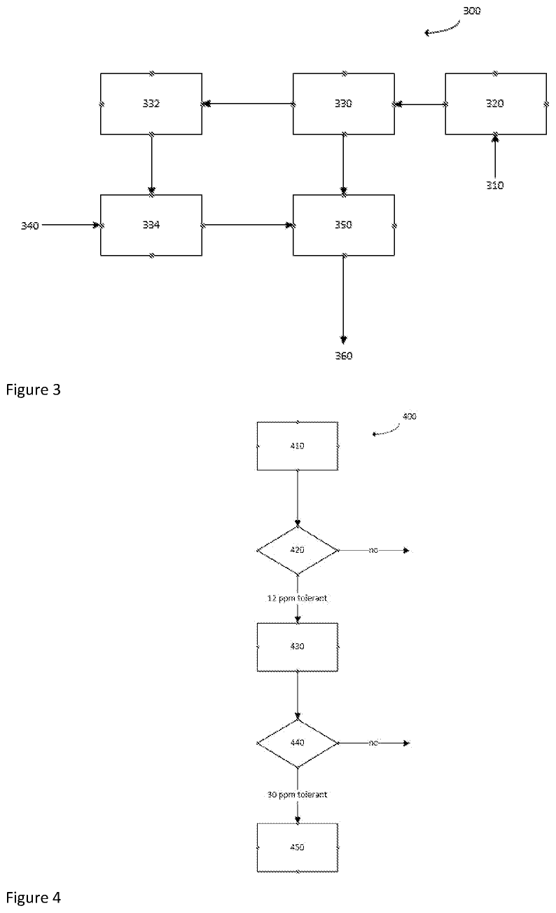

With reference to FIG. 3, according to an embodiment, there is provided a process 300 for producing a biofertilizer composition for soil inoculation and/or foliar application.

The methodology disclosed herein, provides in an embodiment, a solution to a pressing situation impacting food plants in general is illustrated by, for example, banana production, and more generally addresses the problem of sustainable production of formulations of soil microbiology on the farm proven to address these problems, produced using a variety of organic substrates, such that these formulations are inexpensive enough the farmer can apply often through affordable means, including irrigation water. Such application frequency allows the applied microbiology to dominate the soil microbiome and the biome that exists on the stalks and leaves when applied as a foliar.

Sustainable approach to treat Fusarium oxysporum f. sp. cubense infected bananas and contaminated soil. Fusarium oxysporum f. sp. cubense is a fungal plant pathogen that causes Panama disease of banana (Musa), also known as fusarium wilt of banana. The pathogen can be spread in soil, water and by transfer from farming machinery. Banana plants infected with F. oxysporum f. sp. cubense illicit an immune response that causes the plant to release a gel followed by the formation of tylose cells that block the vascular vessels in the plant, restricting the movement of water and nutrients. Early in the infection of the plant, the fungi affect the tips of the feeder roots and later moves into the rhizome.

Several groups have shown that the soil microbe, Pseudomonas fluorenscens, and several species of Bacillus, including B. subtilis, when added around the roots of a banana plant, can reduce Fusarium wilt by 79%. Mohandas et al observed that Banana rhizome treated with Pseudomonas had “massive depositions of unusual structures at sites of fungal entry”, “which clearly indicated that bacterized root cells were signaled to mobilize a number of defense structures for preventing the spread of pathogen in the tissue.” They also observed a 72% reduction of Fusarium when treated with just Pseudomonas. The key innovation is that these microbes have been interacting with plant rhizomes for millennia and add a protective benefit to the plant.

Another threat to the health of the banana are parasitic roundworms called nematodes that eat the roots of the plant. Nematode proliferation can disrupt nutrient and water uptake, delay growth and cause banana plants to topple over. Nematodes account for the direct loss of approximately 19% of total production of bananas.

There is research that shows that the same microbes that can protect banana plans from Fusarium wilt, also protect plants from nematodes. Species of Bacillus, in particular B. subtilis and B. megaterium are strongly implicated in reducing nematode numbers. Abd-El-Khair et al showed that root associated B. subtilis and P. fluorescens contributed significantly to reduced nematode numbers of nematodes (˜82% reduction) while also increasing plant growth by up to 99% as compared to uninoculated control.

Bacteria are crucial for the overall health of plants. Bacteria contribute to plant health through nutrient cycling and through the complex role they play to protect the rhizome from pathogens and soil parasites.

Biofertilizers containing beneficial microbes grown on synthetic media have been demonstrated to be beneficial to plant growth and health and their use reduces costs, fertilizer use and energy, however generating bacteria off site is expensive to process and ship active microbes to individual agriculture sites. The innovation proposed is to use agricultural by-waste as a natural substrate on which to culture the beneficial microbes that will be used for soil inoculation and foliar application and to grow the microbes in continuous culture on or close to where the microbes would be used.

Continuous culture is critical in applications such as proposed here because of the need to replace the current population of soil microbes that are weakening and protect from those preying on the plant. This is because introduced microbes initially must out compete the native microbes and may need to be reapplied. Culture on site using by-waste, specially prepared to be effective in growing only the selected beneficial microbiology in large enough volumes and concentration, is necessary to allow frequent applications. The cost and methodology of production achieves the lowest possible cost while simultaneously increasing yields.

As shown in FIG. 3, by-waste 310 is macerated 320 and placed inside a closed, vented tank 330 with clean water, then heat is applied to increase the temperature to not less than 165 F or higher than 212 F where it is held for a minimum of one minute before being allowed to cool to between about 100 F and about 105 F.

A portion of the clear water is drawn off and sent to a separate small unit 332 and another portion is drawn off and sent to a large tank 350. In the small unit 332 the water is heated to between 165 F and 185 F and held for one minute before being cooled to between 100 F and 105 F and added to a small tank 345 where a selected starting culture 340 was added to and then held. The small tank 334 is filled over time and additional concentrated, select culture 340 is added. The culture in the small tank is held until a specific concentration of the culture in active form is attained, then a portion of this small tank is added to a large tank 350. This process is repeated until the culture in the large tank has attained an estimated concentration of active culture between 1×10e6 and 1×10e9 cfu/ml when it is ready to apply to the soil 360.

This sustainable approach will advance most effective method to reduce the effects of and prevent future infection of Fusarium, reduction of damage due to nematodes while increasing yields and replacing chemicals and thereby benefiting the environment.

According to an embodiment, there is provided a method that uses a foot bath that incorporates biosafety level soil microbes that can rapidly degrade livestock manure along with environmental isolates (from the genus Streptomyces) which produce tetracycline and from streptomycin, which is produced by the soil microbe, Streptomyces griseus. The mixture of facultative anaerobic microbes is chosen based on the best performing biosafety level 1 microbes to degrade the target manure. The selection of the antibiotic-producing microbes is based on the effectiveness of producing their antibiotic compounds that are shown to be most effective in eliminating the bacterial load on contaminated hooves. Unlike copper sulfate, which accumulates and persists in the environment, tetracycline and streptomycin can be degraded by environmental microbes and though chemical processes such as hydrolysis and photolysis.

With reference to FIG. 4, according to an embodiment, there is provided a process 400 of producing copper tolerant/adapted microbes adapted to degrade copper contaminated organic waste. In broad aspects, the method detailed below increases the copper resistance in Bacillus and similar genera gradually from a base copper level to an elevated copper level and so that adapted Bacillus or similar copper intolerant microbes can digest copper contaminated waste. This same technique can be used on other genera and with other contaminating metals. The base copper level is selected based on observed ranges of concentrations of copper in the contaminated site (e.g. a copper contaminated-barn housing animals and contaminated animal waste) which is selected to as the elevated copper level in which the copper adapted microbes are expected to be used. Hence, the base copper level is a level of copper selected to acclimate the intolerant microbes up to the eventual environment in which they are intended to be used.

In one aspect, the method of producing copper tolerant microbes adapted to degrade copper contaminated organic waste, the method comprises at step 410 culturing copper intolerant microbes in a solid growth medium containing a base copper level containing from about 12 ppm to less than about 30 ppm copper at about 35 degrees for an incubation time of about 6 to about 24 hours. At step 420 selecting 12 ppm tolerant microbes and growing the 12 ppm tolerant microbes in a liquid nutritional medium. At step 430 harvesting at least a portion of the 12 ppm tolerant microbes and growing the at least a portion of the 12 ppm tolerant microbes in a solid growth medium containing an elevated copper level containing from at least about 30 ppm copper at about 35 degrees for an incubation time of about 6 to about 24 hours. At step 440 obtaining the copper tolerant microbes by selecting the 30 ppm tolerant microbes and at step 450 growing the 30 ppm tolerant microbes in a MnCl₂-supplemented nutritional medium to sporulation, if desired.

### Example Procedure

1. Reconstituted freeze-dried bacteria. Inoculated liquid NB media.
2. Made glycerol stocks labeled the original strains with (−) to note that they do not have the resistance to Cu.
3. Streaked solid media with each of the reconstituted strains. Incubated at 35 C overnight 4. Made NB agar infused with 12 PPM copper (from CuSO4).
5. Streaked each of the five strains to 12 ppm copper and incubated at 35 C overnight.
6. Grew 12 ppm resistant strains in NB==>made glycerol stock using 50% sterile glycerol (50/50 mixture with bacteria). Stored at −30 C
7. Made 30 ppm copper infused agar media. Streaked plates with the 12-ppm copper adapted bacillus. Picked adapted microbes and inoculated NB. Grew overnight and made glycerol stocks.
8. Grew each strain in 500 ml of NB supplemented with MnCl₂ to sporulation.
9. Collected cells by centrifugation then into 20% isopropyl.

### Example Recipe

A double concentration of SG (Sporulation Agar) was prepared as described in Goldrick, S. and Setlow, P. (1983) Expression of a Bacillus megaterium sporulation specific gene in Bacillus subtilis. Journal of Bacteriology;155(3):1459-62
Per liter of distilled water

| | |
|---|---|
| Difco Nutrient Broth | 16.0 g |
| KCL | 2.0 g |
| MgSO4 (anhyd) | 0.244 g |
| Agar | 17.0 g |

Adjust the pH to 7.2 with 40 ul/L 1 M NaOH. Autoclave and cool to 55 C then add:

| | |
|---|---|
| 1M Ca(NO₃)₂ | 1.0 ml |
| 0.1M MnCL2 * 4 H₂O | 1.0 ml (27.8 g/250 ml) |
| 1 mM FeSO4 | 1 ml |
| Glucose 50% (w/v), filter sterilized | 2.0 ml |

Sporulation data for 30 ppm Cu-Adapted Bacillus after 7 days at 35 C

| | |
|---|---|
| #1 B. subtilis subsp. Inaquosorum | 95% |
| #2 B. subtilis 6051a | 95% |
| #3 B. licheniformis | 95% |
| #4 B. mojavensis | 99% |
| #5 B. megaterium | 95% |

Digestion of copper contaminated dairy cow pressate:
Made media recipe containing (per liter);

| Per liter | Media | |
|---|---|---|
| Chicken Stock | 120 ml | (½ cup) |
| Molasses | 5 ml | |
| Sodium Bicarbonate | 5 g | (0.176 oz) |

| | |
|---|---|
| Miracle Gro (24-8-16) | 15 tsp/0.3 g |

Added 1.34 ml (1/746 inoculation volume (0.94%) of endospore concentrate to 100 ml of media that had been heated for 3 minutes in the microwave to heat shock the endospores. Transferred 50 ml of heat-shocked endospores into 450 ml of fresh media×2 (1-liter inoculated total) at 5:30 PM January 10. Put media on rotary shaker at 80 F until Friday morning at ˜7 AM (37 hours incubation time).
OD reading at 0430 January 11=0.335
Checked OD of cultures at 6 AM January 12=1.118. The culture was 37 hours old.
37-hour old cultures were used to create dilutions of 5:1 and 10:1, including controls at each dilution with no addition of bacteria. The sample jars were placed on a shaker in a room at 80 F and shaken vigorously to introduce oxygen before each sampling.
Collected 50 ml every hour from each sample into pre-weighed aluminum weigh boats then put the samples into the drying oven. Samples were collected for 8 hours.
Observation: The inside of the jars is forming condensation even though the room temperature is 64 F that remained in the uncapped jars and the sediment layer is almost indistinguishable at all concentrations.
Samples were dried for at least 24 hours at 105 C.

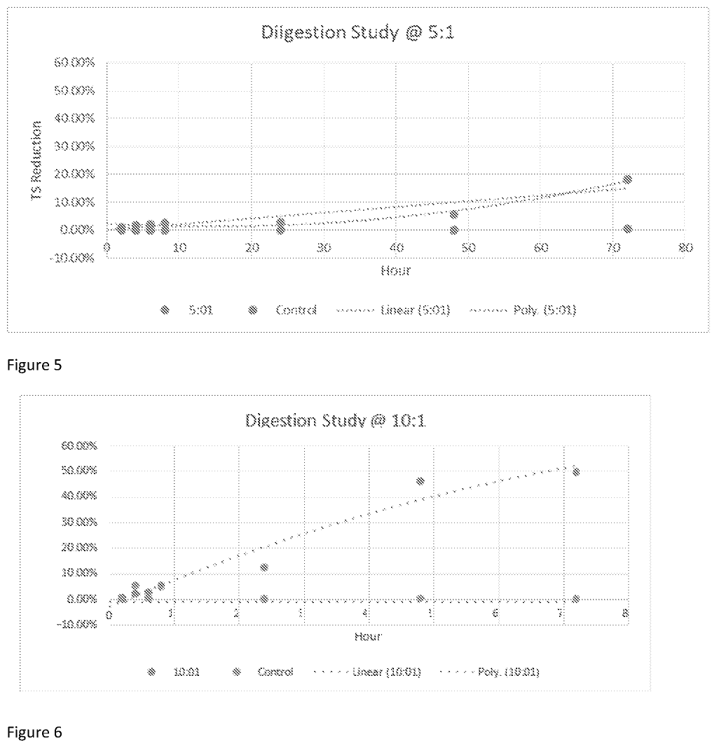

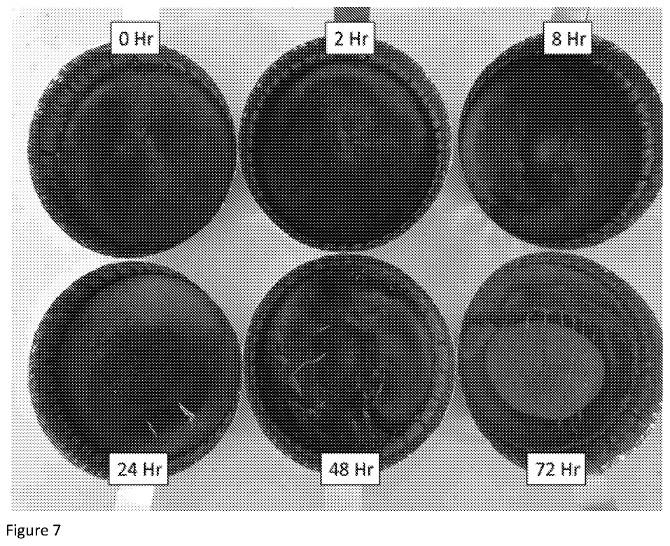

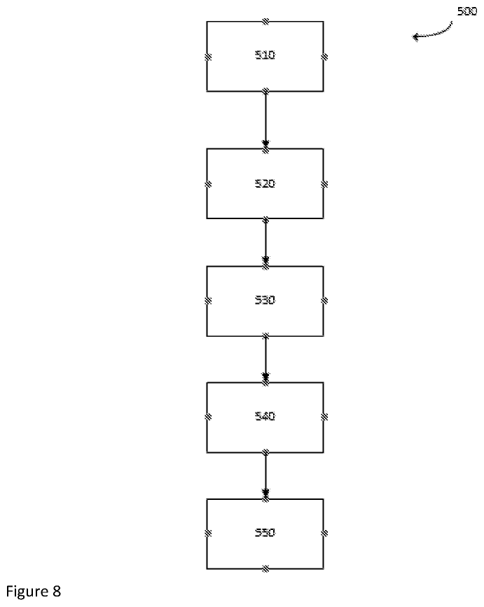

The results of the above are shown in FIG. 5 (digestion study @ 5:1), FIG. 6 (digestion study @ 10:1), and FIG. 7 (10:1 dried foils from 50 ml wet samples).

With reference to FIG. 4, according to an embodiment, there is provided a process 500 for remediating metal-impacted water and/or remediating organic contaminated water. As shown, the method comprises at step 510 flowing metal-impacted water and/or organic contaminated water to an aerated fluidized bed reactor comprising sand and soil-based bacteria to reduce susceptible metals in the water and/or hydrocarbon degrading bacteria to reduce organic contaminants in the water.

At step 520 retaining for a first period sufficient to allow the soil-based bacteria to reduce susceptible metals in the water and/or the hydrocarbon degrading bacteria to remove organic contaminants in the water, and form treated water.

At step 530 flowing clean water, when the treatment time has lapsed, to the first fluidized bed to displace the treated water.

At step 540 flowing the displaced treated water containing any susceptible metals not reduced in the aerated fluidized bed reactor and/or organic contaminants not removed in the aerated fluidized bed reactor to an anaerobic fluidized bed reactor comprising sand and anaerobic or facultative bacteria.

At step 550 retaining for a second period sufficient to allow the anaerobic or facultative bacteria to reduce any susceptible metals not reduced in the aerated fluidized bed reactor and/or remove organic contaminants not removed in the aerated fluidized bed reactor, and form remediated metal-impacted water and/or remediated organic contaminated water.

According to one aspect, the method efficiently reduces toxic metals concentrations. The added benefit of the proposed approach is the simultaneous reduction in nitrogen, sulfur and phosphorous as well as many types of organics.

Sulfate reducing bacteria have been shown to precipitate Cu2+, Fe2+, Zn2+, Ni2+, Cd2+.

Anaerobic reduction has been demonstrated on Hg, Se, and As and is recommended to be the preferred method to remediate coal-impacted water.

MICP has been demonstrated to co-precipitate Ca2+Cu2+, Zn2+, Mg2+Mn2+Cd2+, Co2+, Ni2+, Zn2+, Pb2+, Fe2+, As, Cr, and other cations, as well as many radionuclides.

Some organics, like hydrocarbons can only be removed aerobically while many other polluting organics such as poly- and per-fluorinated (PFAS) compounds can only be removed via anaerobic processes, thus a method that combines each of the biological remediation methods is necessary.

In the proposed method, two or more sequential fluidized beds containing sand would be used and contaminated water would be passed through each of the sequential bed. Aerobic conditions would be maintained, and indigenous soil bacteria would be stimulated to begin the process of MICP to reduce susceptible metals, and the enrichment of hydrocarbon degrading bacteria to degrade susceptible metals. An alternative would be to add specific exogenous bacteria selected to degrade specific compound(s).

At the end of the required resident time for the specific remediation needs, clean water would be injected through the first fluidized bed to push out any remaining metals that had not been precipitated by MICP into the second bed that would be operated under strict anaerobic conditions. Under this step, nitrates, sulfates and susceptible metals would be reduced. As with the first bed, the reductions can be catalyzed by indigenous bacteria or by exogenous bacteria that are added to meet the needs for the remaining metals and organics.

Prior use of MICP has focused upon creating a solid mass in situ while the new methodology is controlled in such a manner as to cause the binding of the target metals to the sand and avoid agglomeration and forming of a solid mass.

Once the sand reaches saturation with targeted metals, it is removed and can be solidified for permanent stability and, in many cases, may be reused as fill material.

While a detailed embodiment of the instant invention is disclosed herein, it is to be understood that the disclosed embodiments are merely exemplary of the invention, which may be embodied in various forms and include most any radius form. Therefore, specific functional and structural details disclosed herein are not to be interpreted as limiting, but merely as a basis for the claims and as a representation basis for teaching one skilled in the art to variously employ the present invention in virtually any appropriately detailed structure. One skilled in the art will readily appreciate that the present invention is well adapted to carry out the objectives and obtain the ends and advantages mentioned, as well as those inherent therein. The embodiments, methods, procedures, and techniques described herein are presently representative of the preferred embodiments, are intended to be exemplary and are not intended as limitations on the scope. Changes therein and other uses will occur to those skilled in the art, which are encompassed within the spirit of the invention and are defined by the scope of the appended claims. Although the invention has been described in connection with specific preferred embodiments, the invention, as claimed, should not be unduly limited to such specific embodiments. Indeed, various modifications of the described modes for carrying out the invention, which are obvious to those skilled in the art, are intended to be within the scope of the following claims.

1. A method for reducing cyanobacteria in a cyanobacteria-contaminated of a body water, the method comprises:
inactivating resident vegetative microbiology from an extract obtained from a cyanobacteria-contaminated body of water to inactivate the resident vegetative microbiology in the extract,
selecting one or more soil-based microbes suitable for growth in the cyanobacteria-contaminated body of water,
growing the one or more soil-based microbes with the inactivated extract to allow the one or more soil-based microbes to adapt to the inactivated extract,
releasing the one or more soil-based microbes into the cyanobacteria-contaminated body of water where the one or more soil-based microbes dominate and reduce cyanobacteria of the cyanobacteria-contaminated body of water.

2. The method of claim 1 wherein the inactivating is by pasteurizing, irradiating, chemically treating and/or mechanically treating the extract.

3. The method of claim 1 wherein the soil-based microbes are capable of degrading toxins from the cyanobacteria and/or are facultative spore-formers with rapid growth rates.

4. The method of claim 1 wherein the soil-based microbes are from Micrococcaceae, Bacillaceae, Pseudomonadaceae, Planococcaceae, or Cellulomonadaceae.

5. The method of claim 1 wherein the releasing is when the one or more soil-based microbes are in a vegetative or active form and capable of assimilating nutrients or chemicals at the cyanobacteria-contaminated body of water.

6. The method of claim 1 wherein the releasing is when the metabolism of the one or more soil-based microbes are most active and in the highest density such that when released into the cyanobacteria-contaminated body of water, the one or more soil-based microbes will require and will consume any nutrients in the cyanobacteria-contaminated of body of water.

7. A method for decontaminating irrigation water and restoring soil contaminated with contaminated irrigation water, the method comprises:
inactivating contaminated irrigation water to inactivate vegetative microbiology to produce inactivated irrigation water,
selecting one or more microbes suitable for outcompeting microbiology in the soil contaminated with the inactivated irrigation water,
growing, under aerobic conditions, the one or more microbes with the inactivated irrigation water to allow the one or more microbes to adapt to the inactivated irrigation water and to inactivate any obligate anaerobic bacteria endospores that may have survived inactivation,
releasing the one or more microbes and the inactivated irrigation water into the soil contaminated with contaminated irrigation water where the one or more soil-based microbes dominate and reduce contamination and restore the soil.

8. The method of claim 7 wherein the inactivating is by pasteurizing, irradiating, chemically treating and/or mechanically treating the extract.

9. The method of claim 8, wherein the method further comprises filtering and/or centrifugation before the growing.

10. The method of claim 7 wherein the one or more microbes are soil-based microbes that promote composting of cellulosic materials in the soil and/or accelerate the breakdown to make additional carbon and nutrients available to the plants or the one or more microbes are one or more of nitrogen-fixing microbes, endophytic microbes, and have the ability to make excess phosphate bioavailable to plants.

11. (canceled)

12. The method of claim 10 wherein the one or more microbes are from Micrococcaceae, Bacillaceae, Pseudomonadaceae, Planococcaceae, or Cellulomonadaceae.

13. (canceled)

14. A biofertilizer composition for soil inoculation and/or foliar application, the composition produced according to a method comprising:
macerating an extract of agricultural by-waste,
inactivating the extract to inactivate resident vegetative microbiology in the extract,
flowing inactivated extract into a holding reservoir and a portion of the inactivated extract into a culture reservoir,
growing in the culture reservoir one or more soil-based microbes with the portion of the inactivated extract to allow the one or more soil-based microbes to adapt to the inactivated extract,
flowing a portion of the one or more adapted soil-based microbes when the one or more adapted microbes are in a vegetative or active form and capable of assimilating nutrients or chemicals into the holding reservoir until the concentration of the one or more adapted soil-based microbes in the holding reservoir is from about 10e6 and 10e9 colony forming units (cfu)/ml.

15. The composition according to claim 12, wherein the method further comprises:
flowing out of the holding reservoir the one or more adapted soil-based microbes when the concentration of the one or more adapted soil-based microbes in the holding reservoir is from about 10e6 and 10e9 cfu/ml,
seeding an amount of the one or more soil-based microbes into the culture reservoir so as to allow the seeded amount of the one or more soil-based microbes to adapt to conditions in the culture reservoir, and
flowing an additional portion of the pasteurized extract into the culture reservoir.

16. The composition according to claim 14 wherein the inactivating is pasteurizing, irradiating, chemically treating and/or mechanically treating the extract.

17. The composition according to claim 16 wherein the inactivating is by pasteurizing by elevating the temperature of the extract to not less than about 165 F and not higher than about 212 F.

18. The composition according to claim 17 wherein the once the temperature of the extract is not less than about 165 F and not higher than about 212 F or not higher than about 185 F, maintaining the temperature of the extract for up to about 60 seconds.

19. (canceled)

20. The composition according to claim 17 wherein after the pasteurizing and before the flowing into the culture reservoir, the temperature of the extract is reduced from to about 105 F or to about 100 F.

21. The composition according to claim 14 wherein the one or more soil-based microbes is one or more of Pseudomonas fluorenscens, B. subtilis, B. megaterium. Micrococcaceae, Bacillaceae, Pseudomonadaceae, Planococcaceae, and Cellulomonadaceae.

22. (canceled)

23. (canceled)

24. Use of the composition according to claim 14 to inoculate soil and/or apply to foliage to restore an agricultural microbiome.

25. The use of claim 24 wherein the agricultural microbiome is an infected banana or other food crops.

26.-67. (canceled)
